(19) United States
(12) Patent Application Publication
(10) Pub. No.: US 2012/0322901 A1
(43) Pub. Date: Dec. 20, 2012

(54) METHOD OF DECONTAMINATION OF POLYOLEFINS

(76) Inventor: Donald W. Hayward, Waterville, OH (US)

(21) Appl. No.: 13/581,061

(22) PCT Filed: Feb. 18, 2011
(86) PCT No.: PCT/US11/25375

§ 371 Date: Aug. 24, 2012

Related U.S. Application Data
(60) Provisional application No. 61/307,628, filed on Feb. 24, 2010.

(51) Int. Cl. C08J 11/06; A61L 2/00

(52) U.S. Cl. 521/47; 521/49.8; 422/5

(57) ABSTRACT

A process for removing a contaminant from post consumer recycled (PCR) plastic is disclosed, the process comprising the steps of comminuting the PCR, to prepare particles having a particle size from about 0.001 inch to about 0.15 inch, wherein the PCR plastic is not polyethylene terephthalate; and driving the contaminant out of the plastic particles by causing the contaminant to diffuse out of the plastic particles.

### CROSS-REFERENCE TO RELATED APPLICATION

This application claims the benefit of priority of U.S. Provisional patent application Ser. No. 61/307,628 filed on Feb. 24, 2010 hereby incorporated herein by reference in its entirety.

### FIELD OF THE INVENTION

This invention relates generally to an improved process for decontaminating post-consumer recycled plastic (PCR plastic) through particle size reduction. More particularly, the invention is directed to a process for removing contaminants which have diffused into the matrix of a plastic container or plastic packaging, which process is conducted after the container is recycled into PCR plastic.

### BACKGROUND OF THE INVENTION

Methods of post-consumer processing of recycled plastics, such as HDPE, PP and other plastics, used to manufacture a variety of useful consumer products, such as flower pots and fence posts, are well-known. Typically, the plastic recycling process utilizes used plastic containers, such as discarded milk containers, which are collected, sorted, washed, and separated from contaminants to yield a relatively clean source of PCR plastic. Additionally, the manufacture of unused but imperfect or damaged molded plastic products results in a considerable amount of plastic waste which manufacturers desire to reuse. The PCR plastic produced by conventional recycling processes is generally in a flake form. The flake is melt processed or further pelletized by an end user.

PCR plastic is typically subjected to a grinding operation in order to put the PCR plastic in a form easier to handle and process. Conventional grinding equipment reduces the PCR plastic to about ⅜ inch (9.5 mm) sized particles or flakes. The grinding is conducted in a manner to ensure that a consistent flake size will be produced, by employing a grate or screen through which the ground material must pass upon exiting the grinder. It is understood in the plastic recycling industry that ⅜ inch flake is generally assumed to mean flakes which have passed through a grate or screen with ⅜ inch openings. Although conventional PCR plastic flakes melt processing and pelletizing equipment is designed to handle ⅜ inch flakes, PCR plastic materials having sizes as large as ½ inch or as small as ¼ inch are also commercially available. The bulk density of ⅜ inch flake PCR plastic generally ranges from about 10 pounds per cubic foot to about 35 pounds per cubic foot.

Similarly, PCR plastic and PCR plastic pellets are generally formed to a standard, uniform size about 0.12 inch (3 mm). The bulk density of such pellets generally ranges from about 20 to about 58 pounds per cubic foot. Typically, PCR plastic melt processing equipment is designed to accept pellets having the above mentioned dimensions and physical characteristics.

The critical aspect for achieving consistently high quality end products utilizing PCR plastic is comprehensive decontamination of the PCR plastic flakes or pellets. Significant decontamination occurs during the washing and sorting of PCR scrap, such as plastic containers, bottles, and other products. Incoming PCR bottles and containers are comminuted to form fragments and to remove loose labels, dirt, and other foreign particles. Thereafter, the mixture of plastics and contaminants is air classified, and the plastics are washed in a hot detergent solution to remove additional label fragments and adhesives therefrom. The washed plastic fragments are then rinsed and placed in a series of flotation baths to separate heavy foreign particles and light foreign particles from the plastics. The remaining plastics are then dried and sold as PCR plastic flakes. Thus, label and other packaging glues, paper, glass, and metals, all of which adversely affect the quality and performance of the finished product, are removed from the PCR plastic.

Of recent concern are the toxic contaminants which may be introduced into a typical PCR plastic processing stream. Examples of such contaminants include pesticides, solvents, herbicides, and chlorinated hydrocarbons which could contaminate the PCR plastic through inadvertent contact during processing or transporting, or by the recycling of bottles and containers which were used by consumers to hold toxic substances.

D. W. Hayward, “Employing RPET in Your Process,” SPE RETEC, Nov. 4, 1994 reports that “clean” RPET flakes can still contain residual contaminants in concentrations as high as 4%, and that such contaminants could include toxic contaminants. These sources of contamination are of concern to those who desire to incorporate PCR plastic into new containers for food-contact use,

Polyolefin plastics, such as polyethylene and polypropylene, are known to absorb more volatile contaminants than those absorbed by polyethylene terephthalate (PET), and polyolefin plastics melt at a significantly lower temperature. PET melts at 260° C. while HDPE melts at only 130° C. These are at least two of the reasons why it is more difficult to remove volatiles from HDPE and PP compared to PET during a recycling process. Even after washing, melting, and pelletizing processes, polyolefins may have a strong odor which makes it unsuitable for use in forming packaging, such as shampoo bottles or soap containers, as the objectionable odor from the plastic may be transferred to the product. This has historically made it difficult to achieve a level of purity in recycled polyolefins which meets United States FDA and EU standards for use of recycled polyolefins in food containers.

Much prior art has been directed towards improving the organoleptic properties of recycled polyolefins. For instance, U.S. Pat. No. 5,350,788 for METHOD FOR REDUCING ODORS IN RECYCLED PLASTICS AND THE COMPOSITIONS THERETO, teaches the addition of polyethylene imines to recycled polyolefin to remove aldehyde odors.

Aldehydes from rancid fats and oils, such as from dairy products, are often associated with the homopolymer polyethylene of recycled milk jugs. In the WRAP (Waste and Resources Action Programme) process, HDPE from recycled milk jugs is heated, subjected to a series of vacuum chambers for an extended period of time, then melted and extruded in a vacuum-vented extruder, and pelletized. Other HDPE decontamination processes expose HDPE flakes to a fluidized bed hot air stream for several hours.

Since RPET decontamination typically takes place at temperatures of 180° C. or higher, temperatures at which both PP and HDPE would melt, the process disclosed in commonly-owned U.S. Pat. No. 5,899,392 was not considered applicable to polyolefins and other low-temperature melting polymers, Additionally, the aldehyde and other volatile contaminants present in recycled polyolefins are not typically encountered when recycling PET plastics and containers. The volatile chemicals that cause persistent odor problems in polyolefin recycling are not common in PET recycling. Therefore, the method of the '392 patent was not considered for polyolefins and other lower-temperature melting polymers. It was surprisingly found that the method of the '392 patent is effective at removing volatiles from recycled polyolefin plastics.

With regard to the possibility that toxic contaminants could be contained in PCR plastic designed for food-contact use, the United States FDA has set protocols for the levels of such contaminants, and the United States FDA has established surrogate and concentration limits to establish the effectiveness of washing and subsequent decontamination processes. Because the United States FDA challenge-testing protocols require that the PCR plastic be introduced to the selected contaminant for as long as two weeks, it is clear that contaminants will diffuse into the polymer matrix of a bottle or container that is subsequently recycled. Accordingly, an effective decontamination method may require that the contaminant be driven back out of the PCR plastic flakes produced from the bottle or container sidewalls, in order to meet the required contaminant concentration limit.

It would be desirable to develop a process for decontaminating PCR plastic to produce PCR plastic having a contaminant level below a level that is acceptable for manufacturing new plastic bottles and containers or other packaging material, including food-grade containers.

### SUMMARY OF THE INVENTION

Concordant and congruous with the present invention, a process for decontaminating PCR plastic to produce PCR plastic having a contaminant level below a level that is acceptable for manufacturing new plastic bottles and containers or other packaging material, including food-grade containers has surprisingly been discovered.

According to one embodiment of the invention, a process for removing a contaminant from post consumer recycled (PCR) plastic comprises the steps of comminuting the PCR, to prepare particles having a particle size from about 0.001 inch to about 0.15 inch, wherein the PCR plastic is not polyethylene terephthalate; and driving the contaminant out of the plastic particles by causing the contaminant to diffuse out of the plastic particles.

### BRIEF DESCRIPTION OF THE DRAWINGS

The above, as well as other advantages of the present invention, will become readily apparent to those skilled in the art from the following detailed description of a preferred embodiment when considered in the light of the accompanying drawings in which:

FIG. 1 is a graph illustrating the rate of decontamination of lindane (an insecticide) at 80° C. as a function of decreasing particle size of a high-density polyethylene (HDPE) plastic;

FIG. 2 is a graph illustrating the decontamination rates of lindane, toluene, chloroform, and tetracosane at 80° C. from polyethylene particles having a particle size of about 2.5 mm; and

FIG. 3 is a graph illustrating the decontamination rates of lindane, toluene, chloroform, and tetracosane at 100° C. from polyethylene particles having a particle size of about 200 μm diameter.

### DETAILED DESCRIPTION OF EXEMPLARY EMBODIMENTS OF THE INVENTION

The following detailed description and appended drawings describe and illustrate various exemplary embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical,

The present invention is directed to a process for removing contaminants from PCR plastic flakes, not typically including PET particles. The PCR plastic flakes are comminuted to prepare extremely small PCR plastic particles and thereafter the volatile contaminant is driven out of the PCR plastic particles. By the term “PCR plastic flakes” as it is used herein is meant generally the commercially available recycled plastic materials such as polyethylene and polypropylene produced by conventional plastic recycling methods, usually in flake form, but which may additionally be in the form of chunks, spheres, pellets, and the like, and which are generally made available in bulk and having a particle size from about ¼ inch to about ½ inch for subsequent melt processing operations.

A typical particle of ⅜ inch PCR plastic flake, such as a flake of HDPE of dimensions 9 mm×9 mm×0.305 mm, exhibits a surface area to volume ratio (SA:V) of about 7002 m⁻¹ A particle of HDPE with a diameter of about 200 μm (about 0.0079 inch) has a SA:V of about 30,000 m⁻¹.

Contaminants which have penetrated the PCR plastic flake matrix can only diffuse out at the surface of the PCR plastic flake. Contaminants which have diffused deeply into the PCR plastic flake matrix generally cannot diffuse out of the flake between the time the PCR plastic flake is produced in the conventional recycling process and the time the PCR plastic flake is utilized in a melt processing operation for producing a new plastic article. According to the present invention, PCR plastic flakes are comminuted by any conventional means to prepare PCR plastic particles having an average particle size from about 0.001 inch to about 0.15 inch. Preferably, the particle size ranges from about 0.005 inch to about 0.05 inch. This is a substantial reduction in the size of the individual PCR plastic flakes, and will allow any contaminant contained within the PCR plastic flakes to be driven out more easily and quickly. For example, a particle of HDPE (high-density polyethylene) having a particle size of about 2.5 mm and a concentration of chloroform of 1,000 ppm requires 280 minutes of diffusion time at 100° C. for the level of chloroform to fall to a concentration of 5 ppm. By contrast, a particle of HDPE having a concentration of chloroform of 1,000 ppm and a radius of about 200 μm requires less than 3 minutes to be driven to the same 5 ppm concentration level, all other process and particle parameters being equal. Thus, PCR polyolefin plastic flakes may be decontaminated by the inventive process, which includes the step of particle size reduction, without the need for elaborate or exotic means such as twin-screw compounding, vacuum extraction, or extremely long residence times such as are taught in the prior art.

Following comminution of the PCR plastic flakes, the resultant PCR plastic particles are subjected to a process designed to drive the contaminants out of the particles. As is well-known, this may be accomplished by merely air drying the PCR plastic particles (passing a stream of a gas, preferably air, over and through the particles) at room temperature. The time required to achieve the substantial elimination of contaminants from the PCR plastic particles is much less than the time that otherwise would be required to achieve the same elimination of contaminants from an equal mass of ⅜ inch PCR plastic flakes, utilizing the same conditions. Alternatively, the comminuted PCR plastic particles may simply be allowed to reside in bulk at standard conditions until the contaminants have diffused out of the particles. Moreover, the PCR plastic particles may be heated in a conventional manner which will accelerate the diffusion of the contaminants out from the particles. Also, the PCR plastic particles may be placed in a liquid solution that can leach the contaminants out from the particles. These, as well as other conventional methods may be used to drive the contaminants out from the PCR plastic particles; but in each case, the time required will be substantially less than would otherwise be required to affect the same level of decontamination upon an equal mass of the common commercially available PCR plastic flakes.

Although the present invention focuses primarily upon the use of particle size reduction to decontaminate the PCR plastic particles, other benefits may be realized from employing the particle size reduction step. Comminution of the PCR plastic particles also improves the drying performance of polyolefins and the drying and solid-stating of PCR polycondensation polymer plastics, like nylon and polycarbonate.

Thus, although the inventive process is described and claimed as one for removing a contaminant from PCR plastic flakes, most specifically polyolefins, it is contemplated that the described and claimed process may additionally be employed with polycondensation polymers to improve and accelerate a solid-stating process, and may simplify and accelerate drying processes.

### EXAMPLE

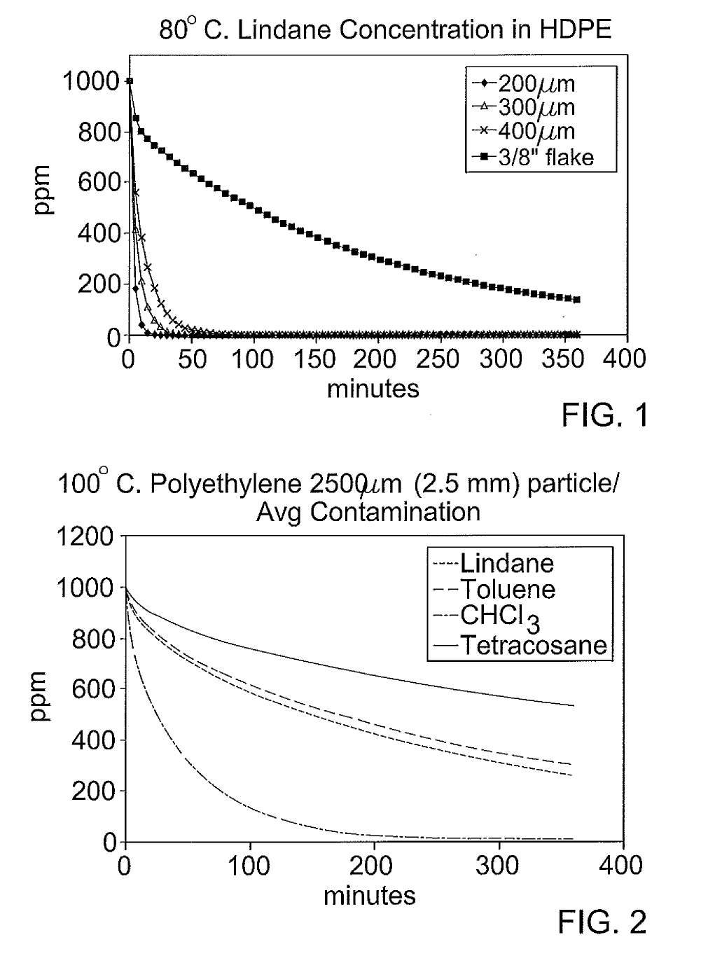

FIG. 1 graphically illustrates a level of decontamination versus time of lindane from HDPE at 80° C. Four samples of HDPE were decontaminated: HDPE particles having a particle size of about 200 μm, HDPE particles having a particle size of about 300 μm, HDPE particles having a particle size of about 400 μm, and HDPE particles having a particle size of about ⅜ inch. As shown, the decontamination rate of the ⅜ inch, uncomminuted HDPE flake is substantially slower than the rate of particles having a particle size of between about 200 μm and about 400 μm,

FIG. 2 graphically illustrates decontamination rates of HDPE plastic particles having a size of about 2.5 mm (0.1 inch) using methods known in the prior art. The rates of decontamination at 100° C. are plotted for example surrogate contaminants lindane, toluene, chloroform (CHCl₃), and tetracosane (H(CH₂)₂₄H). These surrogate contaminants represent a broad range of possible contaminants in the post-consumer plastic recycling stream to which recycled plastic packaging material may potentially be exposed. In particular, chloroform and tetracosane are considered reasonable surrogates for the aldehydes from rancid fat and oil which cause objectionable odors in recycled polyolefins, like HDPE milk jugs.

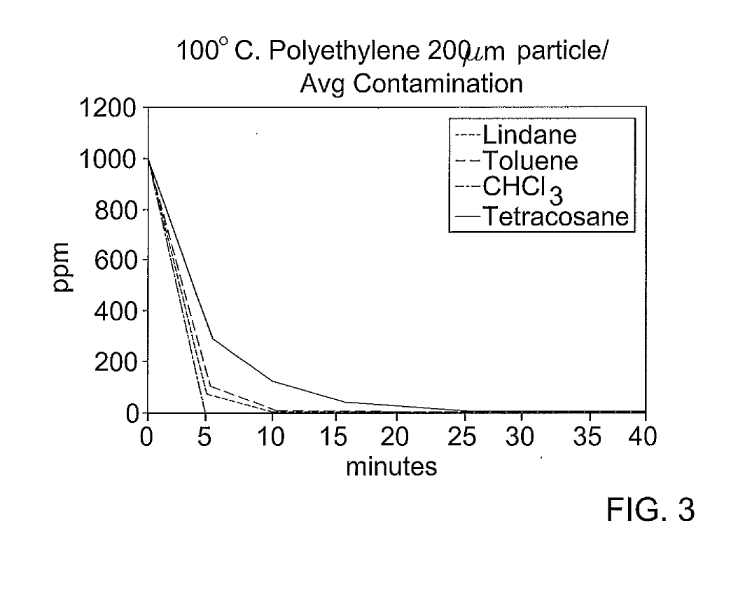

Referring to FIG. 3, HDPE plastic flake comminuted to an average particle size of 200 μm is decontaminated at the same conditions employing the same surrogate contaminants. The extremely small particles are completely decontaminated in less than 30 minutes, compared to the 2.5 mm flake illustrated in FIG. 2, which was decontaminated after 6 hours.

The process for removing contaminants from PCR polyolefin plastic flakes described hereinabove is generally disclosed in terms of its broadest application to the practice of the present invention. Occasionally, the process conditions as described may not be precisely applicable to each contaminant I PCR plastic combination included within the disclosed scope. Those instances where this occurs, however, will be readily recognized by those ordinarily skilled in the art. In all such cases, the process may be successfully performed by conventional modifications to the disclosed process.

The invention is more easily comprehended by reference to specific embodiments recited hereinabove which are representative of the invention. It must be understood, however, that the specific embodiments are provided only for the purpose of illustration, and that the invention may be practiced otherwise than as specifically illustrated without department from its spirit and scope.

From the foregoing description, one ordinarily skilled in the art can easily ascertain the essential characteristics of this invention and, without departing from the spirit and scope thereof, can make various changes and modifications to the invention to adapt it to various usages and conditions.

1. A process for removing a contaminant from post consumer recycled (PCR) plastic comprising the steps of
comminuting the PCR plastic to prepare plastic particles having a particle size from about 0.001 inch to about 0.15 inch, wherein the PCR plastic is not polyethylene terephthalate; and
driving the contaminant out of the plastic particles by causing the contaminant to diffuse out of the plastic particles.

2. The process of claim 1, wherein the PCR plastic is a polyolefin plastic.

3. The process of claim 1, wherein the PCR plastic is selected from a group consisting of polyethylene, polypropylene, polystyrene, and polylactic acid.

4. The process of claim 1, wherein the PCR plastic is comminuted to prepare plastic particles having a particle size from about 0.001 inch to about 0.15 inch.

5. The process of claim 4, wherein the PCR plastic is comminuted to prepare plastic particles having a particle size from about 0.005 inch to about 0.05 inch.

6. The process of claim 5, wherein the PCR plastic is comminuted to prepare plastic particles having a particle size of about 0.0079 inch.

7. The process of claim 1, wherein the step of driving the contaminant out of the plastic particles comprises passing a stream of a gas over and through the particles.

8. The process of claim 1, wherein the step of driving the contaminant out of the plastic particles comprises a step of allowing the plastic particles to reside in bulk for a period of time sufficient for substantially all of the contaminant to diffuse out of the plastic particles.

9. The process of claim 1, wherein the step of driving the contaminant out of the plastic particles comprises a step of heating the plastic particles.

10. The process of claim 1, wherein the step of driving the contaminant out of the particles comprises a step of immersing the plastic particles in a solution capable of leaching the contaminant out of the particles.

11. The process of claim 1, wherein the contaminant is an aldehyde from one of a fat and an oil.

12. The process of claim 1, wherein the PCR plastic is a polycondensation polymer.

13. The process of claim 1, wherein the polycondensation polymer is one of a nylon and a polycarbonate.

14. The process of claim 1, wherein the contaminant provides an odor.

15. A process for decreasing the time to remove an odor from a post consumer recycled (PCR) plastic comprising the steps of
comminuting the PCR plastic to prepare plastic particles having a particle size from about 0.001 inch to about 0.15 inch, wherein the PCR plastic is not polyethylene terephthalate; and
driving the odor out of the plastic particles by causing the odor to diffuse out of the plastic particles.

16. The process of claim 15, wherein the odor comprises an aldehyde from one of a fat and an oil.

17. The process of claim 15, wherein the step of driving the odor out of the plastic particles comprises one of:
passing a stream of a gas over and through the particles;
allowing the plastic particles to reside in bulk for a period of time sufficient for substantially all of the contaminant to diffuse out of the plastic particles;
heating the plastic particles; and
immersing the plastic particles in a solution capable of leaching the odor out of the particles.

18. A process for reducing an odor in a plastic container or plastic packaging, the odor caused by a contaminant that has diffused into a matrix of the plastic container or plastic packaging, the plastic container or plastic packaging subsequently recycled into a post consumer recycled (PCR) plastic, the process comprising the steps of:
comminuting the PCR plastic to prepare plastic particles having a particle size from about 0.001 inch to about 0.15 inch, wherein the PCR plastic is not polyethylene terephthalate; and
driving the odor out of the plastic particles by causing the odor to diffuse out of the plastic particles.

19. The process of claim 18, wherein the odor comprises an aldehyde from one of a fat and an oil.

20. The process of claim 18, wherein the step of driving the odor out of the plastic particles comprises one of:
passing a stream of a gas over and through the particles;
allowing the plastic particles to reside in bulk for a period of time sufficient for substantially all of the contaminant to diffuse out of the plastic particles;
heating the plastic particles; and
immersing the plastic particles in a solution capable of leaching the odor out of the particles.
